(19) United States
(12) Patent Application Publication
(10) Pub. No.: US 2002/0019004 A1
(43) Pub. Date: Feb. 14, 2002

(54) Method for isolating molecules, cells and other particles which are specifically bound to a large particle

(76) Inventor: Albert Orfao, Salamanca (ES)

Correspondence Address: CROWELL & MORING, L.L.P., INTELLECTUAL PROPERTY GROUP, P.O. BOX 14300, WASHINGTON, DC, 20044-4300, US

(21) Appl. No.: 09/920,727

(22) Filed: Aug. 3, 2001

Related U.S. Application Data
(60) Provisional application No. 60/222,651, filed on Aug. 3, 2000.

(51) Int. Cl. C12Q 1/68; G01N 33/567; G01N 33/545

(52) U.S. Cl. 435/6; 435/7.21; 436/531

(57) ABSTRACT

A method for isolating molecules, cells and other particles which are specifically bound to a large particle. It includes: 1) incubation of the sample with one or more sets of large particles which are able to specifically bind/capture a large number of molecules, cells or other particles, 2) analysis of each individual large-capturing particle mixed with the sample, and 3) sorting the large particles containing specifically-bound molecules, cells or other particles.

This application claims the benefit of priority of provisional application Ser. No. 60/222,651, filed Aug. 3, 2000.

### BACKGROUND AND SUMMARY OF THE INVENTION

The present invention relates to a method for isolating molecules, cells and other particles which are specifically bound to a large particle. It includes: 1) incubation of the sample with one or more sets of large particles which are able to specifically bind/capture a large number of molecules, cells or other particles, 2) analysis of each individual large-capturing particle mixed with the sample, and 3) sorting the large particles containing specifically-bound molecules, cells or other particles.

Although not exclusively, this invention is mainly related to a procedure for isolating molecules (i.e., DNA, mRNA, proteins, peptides, etc.), cells or other particles (i.e., chromosomes, mitochondria, zymogen granules, cell membranes, etc.) which are specifically bound to a large (i.e., 500 mm) particle by means of passing the sample where the molecules, cells or other particles to be sorted are present through a chamber containing the large-capturing particles or vice versa. Alternatively, the sample containing the elements to be sorted can be directly incubated with the large binding/capturing particles. Subsequent sorting of the large-capturing particles containing molecules, cells or other particles bound to their surface is performed in a large particle-sorting flow cytometry-based instrument. This method is particularly suited for the isolation of molecules, cells and other particles present at relatively low concentration in large volumes of sample with minor manipulation of the sample itself. Therefore it represents a quick, cost effective, sensitive and specific method for the isolation of rare molecules, cells and other particles from a large volume of sample.

Enrichment and purification of specific molecules, cells or other particles present at relatively low concentrations in large sample volumes have long represented a major challenge for basic and applied researchers in the area of cell biology. Accordingly, in the past different methods have been developed for the purification of different types of molecules, cells and other particles. The development of these methods has been pushed forward by different goals: 1) the specific identification of the presence or absence of the molecules/particles of interest in a given sample, 2) their quantitative evaluation once identified, and 3) their specific characterization; additionally, interest on the specific identification of a given molecule, cell or particle relates to the possibility 4) to deplete it from the sample under analysis. Although it might happen that the identification, quantitation and characterization or even the depletion of molecules/particles from a given sample might not need their physical isolation, it is not uncommon that the molecules/particles of interest would need to be sorted out from the rest of the sample in order to be able to confirm their presence, measure their amounts, specifically characterize them with different tools or be sure that they have been completely and specifically eliminated from the initial sample.

In the past many different methods have been developed to sort out molecules, cells or other particles from a given sample. At present, the most frequently used methods for the isolation of cells include flow cytometry-based cell sorting, immunomagnetic procedures including those using superparamagnetic particles and free-flow magnetophoresis, immuno-labeled high density particles and panning approaches, together with density gradient centrifugation and centrifugal elutriation, among others. For the isolation and purification of different biological molecules, single- or multi-dimensional electrophoresis, chromatography based techniques (including HPLC and FPLC), density gradient centrifugation, precipitation or affinity based purification approaches are frequently used. Basically, these approaches can be divided into two major groups of methods: bulk sorting procedures and single molecule/cell based methods. The former usually allows for the purification of large amounts of one or more specific type of molecule/particle in a relatively short period of time, but it is associated with both a relatively low purity and/or recovery and extensive manipulation of the initial sample to a level that it would prevent its further use for other purposes. In contrast, the latter group of techniques usually allows for a very high purity but at the expense of using large periods of time to obtain relatively low amounts of pure isolated molecules, cells or other particles of interest. The limitations and disadvantages of both groups of approaches to sort out the molecules, cells or other particles of interest with a high purity and/or recovery become even greater once the molecules, cells or other particles of interest are present in the sample at extremely low frequencies/concentrations.

As an example, the use of single-cell flow cytometry-based sorting procedures would not allow the specific sorting of cells present in a sample at frequencies below the sensitivity detection limit of the instrument which have been claimed to be around 10⁻⁵ to 10⁻⁷. In a similar way, the polymerase chain reaction would have problems identifying an mRNA present in a specific cell population which represents less than 1 cell in 10⁻⁶ to 10⁻⁷ cells in a given sample. In order to be able to increase the sensitivities of these methods they are typically applied to samples which have already undergone through initial enrichment steps for the molecules, cells or other particles of interest. However, such preparative steps are usually associated with lower recovery rates together with increased sample manipulation preventing further use of the original sample for other purposes. Even more, the methods used in these preparative steps also show important limitations whenever management of large volumes of sample (i.e., more than 50 mL) are needed, unless extensive sample preparation/manipulation is performed in advance.

Therefore, at present none of the available methods for the specific isolation of molecules, cells or other particles of interest from a sample allows for the specific isolation of a pure fraction of the molecules, cells or other particles of interest present in large volumes of sample at rather low frequencies and/or concentrations in the absence of extensive manipulation of the sample.

Thus, one of the aims of this invention is to propose a solution for the isolation and purification of molecules, cells or other particles present at either low concentrations or low frequencies that requires the use of large sample volumes to obtained enough amounts of the molecules/particles of interest.

Another aim of the invention is to provide a method to purify these molecules/particles in the absence of extensive sample manipulation procedures which would allow the use of the rest of the sample for other additional purposes.

The procedure of this invention comprises: 1) the incubation of the sample containing the molecules, cells or other particles of interest with one or more sets of large particles which are able to specifically bind/capture a large number of molecules, cells or other particles of interest present in the sample, 2) the analytical evaluation of each of the large-capturing particles mixed with the sample, and 3) sorting of the large particles containing specifically-bound molecules, cells or other particles from both the rest of the sample and the large particles which did not bind to the molecules/particles of interest.

Large capturing particles may be made of different materials or combinations of materials including latex, polystyrene, methacrylate, activated-aldehydes, polyethyleneglycol, and acrolein among others and they may also show different shapes. The large-capturing particles may also be of different intensities (i.e., for isolation of cells, molecules and other particles in body fluids, densities of between 1.06 and 1.07 are preferred) and sizes (i.e., between 200 um and 1 mm, in principle the larger the better). Additionally the more resistant to centrifugation and sedimentation procedures the large-capturing particles are, the better it is. Large-capturing particles should display high amounts of reactive groups on their surface (i.e., COOH groups that allow the interaction of the particle surface and immunoglobulin molecules at high pH) that could be either easily blocked or inhibited (i.e., by changing pH) in order to avoid to a maximum the possibility of non-specific reactions to occur latter on.

In order to specifically bind molecules, cells or other particles of interest, the large-capturing particles are covered with or bound to specific antibodies, DNA oligonucleotides or other probes specific for the molecules, cells or other particles of interest; for this purpose different chemical ligations such as adsorption and covalent binding can be used. Each set of large-capturing particles and each individual particle contained in it may be composed of particles which can bind one or more types of molecules, cells and/or other sample particles.

The incubation of the sample with a set of the large-capturing particles can be performed by any of the following methods: 1) directly mixing the set of large-capturing particles with the sample containing the molecules/particles of interest, 2) passing the sample one or more times through a chamber containing the large-capturing particles and, 3) passing the set of large-capturing particles one or more times through the sample containing the molecules, cells or other particles of interest to be isolated.

The sample can be simultaneously or sequentially incubated with one or more sets of large-capturing particles, each of them being directed to the binding and isolation of a specific molecule(s), cell(s) or particle(s) contained in the sample.

The specific isolation of the large-capturing particles bound to the molecules, cells or other particles of interest present in the sample is achieved by passing the particles through a large-particle sorting device such as the COPAS (Union Biometrica, Boston, Mass., USA) flow cytometry instrument. Distinction between large particles bound to molecules, cells or other type of sample particles from the unbound ones is performed on the basis of fluorescence specific stainings (i.e., fluorochrome-conjugated monoclonal antibodies directed against captured cells or molecules) and scatter properties (i.e., low angle or 90° angle light scatter). During sorting of large-capturing particles bound to cells/molecules of interest, these particles can be sorted into different layers (i.e., Petri dishes, 24-well culture plates, etc.).

The molecules, cells or other sample particles of interest can be detached from the large-capturing particles after they have been sorted using different well-established procedures and they can be used afterwards for different purposes.

As compared to current high-speed single-cell sorters, the. method of the present invention, among other advantages, substantially decreases the time needed for the purification of molecules, cells and other particles present at low frequencies or concentrations, in a large volume of sample. Additionally, it can be used in the absence of extensive sample manipulation which assures the possibility of further using both the depleted sample and the isolated molecules, cells or other particles, for other additional purposes.

The invention can be used for both normal and pathologic samples obtained either exvivo or processed in vitro, for all purposes in which isolation/depletion of molecules, cells or other particles from a sample is required for research, diagnostic, prognostic and therapeutic purposes.

The explanation of the method for the isolation of molecules, cells and other particles of interest is illustrated below with an example which does not limit its impact.

### EXAMPLE

Material and Methods:

Samples: Peripheral blood was obtained from 10 female patients diagnosed as suffering from breast cancer, prior to curative surgery. All patients included in this study showed over expression of the Her2-neu oncogene in fine-needle aspiration diagnostic samples. In all cases, a minimum of 50 mL of ₖ₃EDTA-anticoagulated peripheral blood was obtained by venipuncture. All samples were processed within a period of 6 hours after they were obtained and they were maintained at room temperature until processed. During this period, samples were allowed to sediment.

Sample preparation: All ₖ₃EDTA-anticoagulated peripheral blood samples were centrifuged at 540 g for 5 minutes (room temperature), the supernatant (plasma) was discarded in order to eliminate the soluble Her2-neu protein which could interfere with the binding of Her2-neu+ epithelial cells to the large-capturing particles described below and placed in a separate tube. Afterwards, the cell pellet was resuspended in an identical volume (up to 50 mL/sample) of isotonic saline solution and gently mixed by placing the sample in an end-to-end rotator. Then the sample was incubated with a set of 200 mm beads (Control particles, Union Biometrica, Boston, Mass., USA) which had been previously coated with an anti-Her 2 neu monoclonal antibody (Becton/Dickinson Biosciences, San Jose, Caif., USA) according to well-established procedures. For that purpose the sample was passed through a chamber with a 50 mm cuper net on both its entrance and end connected to the sample stream at both sides. This chamber contained around 10⁴ beads coated with the antiHer2-neu monoclonal antibody. The sample was allowed to pass through this chamber in a discontinuous way by clamping the tubes containing the sample both at the entrance and exit of the chamber. Speed of sample incubation with the beads was of around 1 mL/min.

Afterwards, the depleted sample was collected in a 50 mL Falcon tube (Becton/Dickinson Biosciences, San Jose, Calif., USA), the saline washed out by centrifuging for 10 min at 540 g (room temperature) and resuspended in the plasma. In turn, the chamber containing the beads was washed by passing phosphate buffered saline (PBS) for 5 minutes. Afterwards, the end net of the chamber was opened and the beads allowed to drop into a tube where they remained in suspension in an isotonic buffer (PBS; pH=7.6) (final volume of 20 mL). Then the beads were allowed to sediment by centrifuging for 5 min at 540 g and resuspended in a volume of 300 mL of PBS with 1% freshly prepared paraformaldehyde containing 80 ng/mL of L-alfa-lysophosphatidylcholine (Sigma, St Louis, Mo., USA). Immediately afterwards, 20 mL of an anti-cytokeratin 18 monoclonal antibody directly conjugated to fluorescein isothiocyanate (FITC) was added to the bead suspension and a 15 minute incubation in the darkness was performed (room temperature) to allow the monoclonal antibody to stain the Her2-neu epithelial cells bound to the large-capturing beads.

Sorting of cytokeratin 18+ beads: After this incubation period, 300 uL of PBS containing 1% paraformaldehyde was added to the bead suspension. After a 10 minute incubation period at room temperature, 40 mL of sheath fluid (Union Biometrica) was added to the bead suspension. Then beads were resuspended by gently mixing in an end-to-end rotator. Then the bead suspension was analyzed in a COPAS flow cytometer equipped with an argon ion laser and detectors for both light scatter time of flight and green fluorescence. Beads were run at a speed of around 200 to 250 beads/second. Beads showing green fluorescence at levels higher than background fluorescence were sorted into a Petri dish while remaining beads were sent to the waste container.

1. A process for isolating molecules, cells and other particles which are specifically bound to a large particle comprising:
incubating a sample with at least one set of large-capturing particles each of which are able to specifically bind/capture a large number of molecules, cells or other particles contained in the sample;
analyzing the large-capturing particles mixed with the sample; and
sorting the large-capturing particles containing specifically bound molecules, cells or other particles.

2. A process according to claim 1, wherein said large-capturing particles may be of different sizes, materials, densities, and/or shapes.

3. A process according to claim 1, wherein different types of molecules, cells or other particles can be bound to the large-capturing particles.

4. A process according to claim 1, wherein the large-capturing particles are covered with or bound to specific antibodies, parts of antibodies, oligonucleotides or other types of probes specific for the binding of the molecules, cells and other particles of interest.

5. A process according to claim 1, wherein the sample is simultaneously or sequentially incubated with two or more different sets of large-capturing particles for the isolation/depletion of two or more different types of molecules, cells or other particles from the sample.

6. A process according to claim 1, wherein each set of large-capturing particles can specifically bind one, two or more different types of molecules, cells or other particles from the sample.

7. A process according to claim 1, wherein the incubation of the sample with the large-capturing particles is performed by:
(A) directly mixing the large-capturing particles with the sample containing the molecules, cells or other particles of interest;
(B) passing the sample one or more times through one or more chambers containing the large-capturing particles; or (C) passing the large-capturing particles one or more times through the sample containing the molecules, cells or other particles of interest.

8. A process according to claim 1, wherein the distinction between the large-capturing particles bound to the molecules, cells or other sample particles is based on their scatter, fluorescence or combined scatter and fluorescence properties.

9. A process according to claim 1, wherein the large-capturing particles bound to molecules, cells or other sample particles are sorted into Petri dishes, microtiter plates or other layers.

10. A process according to claim 1, wherein different sample volumes and amounts of large-capturing particles can be used in combination.

11. A process according to claim 1, wherein the molecules, cells or other sample particles of interest are detached from the large-capturing particles after they are sorted.

12. A process according to claim 1, wherein the molecules are selected from the group consisting of DNA, mRNA, proteins, and peptides.

13. A process according to claim 1, wherein the other particles are selected from the group consisting of chromosomes, mitochondria, zymogen granules, and cell membranes.

14. A process according to claim 1, wherein the large particle is selected from the group consisting of latex, polystyrene, methacrylate, activated-aldehydes, polyethyleneglycol, and acrolein.
